(19) United States
(12) Patent Application Publication
(10) Pub. No.: US 2012/0171295 A1
(43) Pub. Date: Jul. 5, 2012

(54) METHODS FOR CRYOPRESERVING AND ENCAPSULATING CELLS

(76) Inventor: Sascha ABRAMSON, Holland Township, NJ (US)

(21) Appl. No.: 13/340,528

(22) Filed: Dec. 29, 2011

Related U.S. Application Data
(60) Provisional application No. 61/428,427, filed on Dec. 30, 2010.

(51) Int. Cl. A61K 35/12; A61K 35/50; A61K 9/14

(52) U.S. Cl. 424/493; 424/93.7

(57) ABSTRACT

Described herein are methods of cryopreserving cells that have been suspended in alginate as well as methods for encapsulating cryopreserved cells that have been suspended in alginate. Further provided herein are cellular compositions comprising cells that have been processed in accordance with the methods described herein.

This application claims priority to U.S. provisional application No. 61/428,427, filed Dec. 30, 2010, the disclosure of which is herein incorporated by reference in its entirety.

### 1. FIELD

Described herein are methods of cryopreserving cells that have been suspended in alginate as well as methods for encapsulating cryopreserved cells that have been suspended in alginate. Further provided herein are cellular compositions comprising cells that have been processed in accordance with the methods described herein.

### 2. BACKGROUND

Cryopreservation is a process in which cells can be preserved by cooling them to low temperatures. At these low temperatures, biological activity, including the biochemical reactions that would lead to cell death under normal conditions, are effectively stopped. As such, cryopreservation provides a valuable means for storing cells for future use. However, certain drawbacks exist in connection with cryopreserving cells, including damage that occurs to the cells during the freezing and/or thawing processes and the need to culture the cells after thawing to ensure that they properly recover. Such drawbacks limit the value of cryopreserved cells, particularly in situations where it is desirable to use the cryopreserved cells immediately or shortly after they have been thawed.

### 3. SUMMARY

It is an objective of the disclosure to provide methods of cryopreserving cells and methods of using cells that have been cryopreserved that solve the above-described problems. Such methods are based, in part, on the discovery that when liquid alginate is added to cells prior to cryopreservation of the cells, the cells can be (i) encapsulated after thawing and then used for their desired purpose without the need to culture the cryopreserved cells after they have been thawed; or (ii) used after thawing in unencapsulated faun without the need to culture the cryopreserved cells after they have been thawed. The methods are additionally based, in part, on the discovery that cryopreserved cells that have been thawed, immediately suspended in alginate, and subsequently encapsulated remain viable and can be used immediately after encapsulation.

As such, in one embodiment, the methods described herein include the addition of liquid alginate to cells so as to form a cell/liquid alginate solution. In some embodiments, a cell/liquid alginate solution is generated and the cells in the cell/liquid alginate solution are cryopreserved, thawed, and encapsulated immediately after thawing and the cells are thereafter used for their desired purpose, e.g., administration to a subject, without any additional culturing, e.g., the cells are used immediately. In some embodiments, a cell/liquid alginate solution is generated immediately after the thawing of cryopreserved cells, which subsequently are encapsulated and are thereafter used for their desired purpose, e.g., administration to a subject, without any additional culturing, e.g., the cells are used immediately. In some embodiments, a cell/liquid alginate solution is generated and the cells are cryopreserved, thawed, and not encapsulated after thawing, rather the cells are used without any additional culturing, e.g., the cells are used immediately.

In one aspect, provided herein are methods for preparing cryopreserved encapsulated cells suitable for administration to subjects immediately after the cryopreserved cells are thawed and encapsulated in alginate. In accordance with this aspect, the cryopreserved encapsulated cells are suspended in liquid alginate prior to their cryopreservation and encapsulated immediately after being thawed. Alternatively, the cryopreserved cells are suspended in liquid alginate immediately after being thawed and subsequently encapsulated. In each case, the encapsulated cells then can be administered to a subject without any additional culturing, e.g., the cells are used immediately after encapsulation.

In a specific embodiment, provided herein is a method for preparing a population of encapsulated cells suitable for administration to a subject, said method comprising: (i) obtaining a population of cells; (ii) adding to said population of cells a cryopreservation solution comprising liquid alginate to produce a cell/liquid alginate composition; (iii) cryopreserving said cell/liquid alginate composition; (iv) thawing said cell/liquid alginate composition; and (v) encapsulating said cell/liquid alginate composition. In certain embodiments, the cells are used after thawing and subsequent encapsulation without any additional culturing, e.g., the cells are used immediately after encapsulation.

In another specific embodiment, provided herein is a method for preparing a population of encapsulated cells suitable for administration to a subject, said method comprising: (i) combining a population of cells and a cryopreservation solution comprising liquid alginate to produce a cell/liquid alginate composition; (ii) cryopreserving said cell/liquid alginate composition; (iii) thawing said cell/liquid alginate composition; and (iv) encapsulating said cell/liquid alginate composition. In certain embodiments, the cells are used after thawing and subsequent encapsulation without any additional culturing, e.g., the cells are used immediately after encapsulation.

In another specific embodiment, provided herein is a method for administering a population of encapsulated cells to a subject, said method comprising: (i) obtaining a population of cells; (ii) adding to said population of cells a cryopreservation solution comprising liquid alginate to produce a cell/liquid alginate composition; (iii) cryopreserving said cell/liquid alginate composition; (iv) thawing said cryopreserved cell/liquid alginate composition; (v) encapsulating said cell/liquid alginate composition; and (vi) administering the encapsulated cell/liquid alginate composition to a subject, wherein said administration is performed in the absence of culturing the cells in said cell/liquid alginate composition after the encapsulation of step (v).

In another specific embodiment, provided herein is a method for administering a population of encapsulated cells to a subject, said method comprising: (i) combining a population of cells and a cryopreservation solution comprising liquid alginate to produce a cell/liquid alginate composition; (ii) cryopreserving said cell/liquid alginate composition; (iii) thawing said cryopreserved cell/liquid alginate composition; (iv) encapsulating said cell/liquid alginate composition; and (v) administering the encapsulated cell/liquid alginate composition to a subject, wherein said administration is performed in the absence of culturing the cells in said cell/liquid alginate composition after the encapsulation of step (iv).

In another specific embodiment, provided herein is a method for preparing a population of encapsulated cells suitable for administration to a subject, said method comprising: (i) obtaining a population of cells; (ii) cryopreserving said cells in a cryopreservation solution; (iii) thawing the cryopreserved cells; (iv) adding to the thawed cells a solution comprising liquid alginate to produce a cell/liquid alginate composition; and (v) encapsulating said cell/liquid alginate composition. In certain embodiments, the cells are used after thawing and subsequent encapsulation without any additional culturing, e.g., the cells are used immediately after encapsulation.

In another specific embodiment, provided herein is a method for preparing a population of encapsulated cells suitable for administration to a subject, said method comprising: (i) cryopreserving a population of cells in a cryopreservation solution; (ii) thawing the cryopreserved cells; (iii) adding to the thawed cells a solution comprising liquid alginate to produce a cell/liquid alginate composition; and (iv) encapsulating said cell/liquid alginate composition. In certain embodiments, the cells are used after thawing and subsequent encapsulation without any additional culturing, e.g., the cells are used immediately after encapsulation.

In another specific embodiment, provided herein is a method for administering a population of encapsulated cells to a subject, said method comprising: (i) obtaining a population of cells; (ii) cryopreserving said cells in a cryopreservation solution; (iii) thawing the cryopreserved cells; (iv) adding to the thawed cells a solution comprising liquid alginate to produce a cell/liquid alginate composition; (v) encapsulating said cell/liquid alginate composition; and (vi) administering the encapsulated cell/liquid alginate composition to a subject, wherein said administration is performed in the absence of culturing the cells in said cell/liquid alginate composition after the encapsulation of step (v).

In another specific embodiment, provided herein is a method for administering a population of encapsulated cells to a subject, said method comprising: (i) cryopreserving a population of cells in a cryopreservation solution; (ii) thawing the cryopreserved cells; (iii) adding to the thawed cells a solution comprising liquid alginate to produce a cell/liquid alginate composition; (iv) encapsulating said cell/liquid alginate composition; and (v) administering the encapsulated cell/liquid alginate composition to a subject, wherein said administration is performed in the absence of culturing the cells in said cell/liquid alginate composition after the encapsulation of step (iv).

In another aspect, provided herein are methods for preparing cryopreserved cells suitable for administration to subjects immediately after the cryopreserved cells are thawed. In accordance with this aspect, the cryopreserved cells are suspended in liquid alginate prior to their cryopreservation and can be administered to a subject immediately after thawing, without the requirement that the encapsulated cells be cultured prior to the administration.

In a specific embodiment, provided herein is a method for preparing a population of cells suitable for administration to a subject, said method comprising: (i) obtaining a population of cells; (ii) adding to said population of cells a cryopreservation solution comprising liquid alginate to produce a cell/liquid alginate composition; (iii) cryopreserving said cell/liquid alginate composition, wherein the cells in the cryopreserved cell/liquid alginate composition can be immediately administered to the subject after thawing.

In another specific embodiment, provided herein is a method for preparing a population of cells suitable for administration to a subject, said method comprising: (i) combining a population of cells and a cryopreservation solution comprising liquid alginate to produce a cell/liquid alginate composition; (ii) cryopreserving said cell/liquid alginate composition, wherein the cells in the cryopreserved cell/liquid alginate composition can be immediately administered to the subject after thawing.

In another specific embodiment, provided herein is a method for administering a population of cells to a subject, said method comprising: (i) obtaining a population of cells; (ii) adding to said population of cells a cryopreservation solution comprising liquid alginate to produce a cell/liquid alginate composition; (iii) cryopreserving the cell/liquid alginate composition; (iv) thawing the cell/liquid alginate composition; and (v) administering the cell/liquid alginate composition to a subject, wherein said administration is performed in the absence of culturing the cells in said cell/liquid alginate composition after the thawing of step (iv).

In another specific embodiment, provided herein is a method for administering a population of cells to a subject, said method comprising: (i) combining a population of cells and a cryopreservation solution comprising liquid alginate to produce a cell/liquid alginate composition; (ii) cryopreserving the cell/liquid alginate composition; (iii) thawing the cell/liquid alginate composition; and (iv) administering the cell/liquid alginate composition to a subject, wherein said administration is performed in the absence of culturing the cells in said cell/liquid alginate composition after the thawing of step (iii).

### 4. DEFINITIONS

The terms “about” or “approximately” mean an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 20%, 15%, 10%, 9%, 8%, 7%, 6%, 5%, 4%, 3%, 2%, 1%, 0.5%, or 0.05% of a given value or range.

As used herein, the term “immediately,” as it relates to the use of the cells described herein, means use of the cells without an intervening culture step. In some embodiments, immediately refers to a length of time that is greater than one week. In some embodiments, immediately refers to a length of time that is greater than six days, greater than five days, greater than four days, greater than three days, greater than two days, or greater than one day. In some embodiments, the term “immediately” refers to length of time that is about 1 day, about 22 hours, about 20 hours, about 18 hours, about 16 hours, about 14 hours, about 12 hours, about 10 hours, about 8 hours, about 6 hours, about 5 hours, about 4 hours, about 3 hours, about 2 hours, about 1 hour, about 30 minutes, about 20 minutes, or about 10 minutes. As used herein, the term “immediately” is not meant to exclude certain steps that may be desired or required before the use of the cells, e.g., dilution of the cells, washing of the cells, and/or storage of the cells (e.g., cold-storage of the cells in an appropriate medium, such as HypoThermosol® (BioLife Solutions, Bothell, Wash.)).

As used herein, the term “alginate” refers to the anionic polysaccharide distributed widely in the cell walls of brown algae. Alginate forms water-soluble salts with alkali metals, such as sodium, potassium, lithium, magnesium, ammonium, and the substituted ammonium cations derived from lower amines, such as methyl amine, ethanol amine, diethanol amine, and triethanol amine. The term “alginate” as used herein encompasses all forms of alginate known to those of skill in the art including, without limitation, calcium alginate, sodium alginate, propylene-glycol alginate, and potassium alginate. Additionally, the term “alginate” as used herein encompasses all terms used by those of skill in the art to describe alginate, e.g., alginic acid and algin.

As used herein, the term “cryoprotectant” refers to any substance that is used to protect biological tissue (e.g., cells) from damage that occurs during freezing, e.g., damage due to ice formation. Exemplary cryoprotectants include, without limitation, glycols (alcohols containing at least two hydroxyl groups) such as ethylene glycol, propylene glycol, and glycerol; dimethyl sulfoxide (DMSO); trehalose; and sucrose. The term “cryoprotectant,” as used herein, is not meant to include alginate.

As used herein, the terms “encapsulation” and “encapsulate” refer to the process by which cells that have been suspended in liquid alginate are enclosed in a semipermeable membrane following exposure of the cell/liquid alginate suspension to divalent cations, e.g., calcium chloride, zinc chloride, copper chloride, and strontium chloride. Encapsulated cells described herein remain viable under both in vitro and in vivo conditions, and also retain their functional and metabolic properties. As used herein, the terms “encapsulation” and “encapsulate” are not meant to encompass the process by which cells are merely frozen in alginate. That is, “encapsulation” as used herein requires the cross-linking of alginate polymers that occurs when alginate is exposed to divalent cations.

As used herein, the term “cryopreservation solution” refers to a solution in which cells may be cryopreserved. Cryopreservation solutions may comprise, without limitation, alginate, cryoprotectants, human serum albumin (HSA), water, protein, salts, buffers, HypoThermosol® (Bio Life Solutions, Bothell, Wash.), and/or dextran. In specific embodiments, cryopreservation solutions do not comprise a cryoprotectant.

As used herein, the term “stem cell” defines the functional properties of any given cell population that can proliferate extensively, e.g., up to about 40 population doublings, but not necessarily infinitely, and can differentiate, e.g., differentiate in vitro, into multiple cell types.

As used herein, the term “derived” means isolated from or otherwise purified. In the context of cells, the term “derived” encompasses cells that are cultured from cells isolated directly from a tissue and cells cultured or expanded from primary isolates.

As used herein, “immunolocalization” means the detection of a compound, e.g., a cellular marker, using an immune protein, e.g., an antibody or fragment thereof in, for example, flow cytometry, fluorescence-activated cell sorting, magnetic cell sorting, in situ hybridization, immunohistochemistry, or the like.

As used herein, the term “isolated cell” means a cell that is substantially separated from other cells of the tissue from which the cell is derived. A cell is “isolated” if at least about 20%, 30%, 40%, 50%, 60%, 70%, 80%, 90%, 95%, or at least about 99% of the cells with which the cell is naturally associated are removed from the cell, e.g., during collection and/or culture of the cell.

As used herein, the term “isolated population of cells” means a population of cells that is substantially separated from other cells of the tissue from which the population of cells is derived. A population of cells is “isolated” if at least about 20%, 30%, 40%, 50%, 60%, 70%, 80%, 90%, 95%, or at least 99% of the cells with which the population of cells, or cells from which the population of cells is derived, is naturally associated are removed from the cell.

As used herein, a cell is “positive” for a particular marker when that marker is detectable above background, e.g., by immunolocalization or by RT-PCR. Conversely, “negative” in the same context means that the particular marker by, e.g., immunolocalization or by RT-PCR, compared to background.

As used herein, the terms “subject” or “patient” are used interchangeably to refer to an animal (e.g., birds, reptiles, and mammals). In a specific embodiment, a subject is a bird (e.g., chicken or duck). In another embodiment, a subject is a mammal including a non-primate (e.g., a camel, donkey, zebra, cow, pig, horse, goat, sheep, cat, dog, rat, and mouse) and a primate (e.g., a monkey, chimpanzee, and a human). In certain embodiments, a subject is a non-human animal. In some embodiments, a subject is a farm animal (e.g., cow, pig, horse, sheep, goat, etc.) or pet (e.g., dog, cat, etc.). In another embodiment, a subject is a human. In another embodiment, a subject is a human infant. In another embodiment, a subject is a human child. In another embodiment, a subject is a human adult.

### 5. BRIEF DESCRIPTION OF THE FIGURES

FIG. 1 shows viability of encapsulated placental stem cells suspended in HypoThermosol® (HT) solution over a span of 7 days at 4° C. and 37° C. “1× cells” corresponds to a cell concentration of 5×10⁵; “4× cells” corresponds to a cell concentration of 2×10⁶.

FIG. 2 shows viability of placental stem cells that were cryopreserved, thawed, suspended in alginate, encapsulated, and stored in HypoThermosol® without intervening culture steps. Viability was assessed at 4 and 24 hours post-encapsulation. “1×” represents 3.75×10⁶ cells/ml; “0.5×” represents 1.88×10⁶ cells/ml; “0.25×” represents 0.94×10⁶ cells/ml.

### 6. DETAILED DESCRIPTION

Provided herein are methods for cryopreserving cells in alginate and methods of administering cells that have been cryopreserved in accordance with the described methods. In certain embodiments, the cells that have been cryopreserved in alginate are encapsulated after thawing. The methods described herein allow for the immediate use of the cryopreserved cells and are thus advantageous over known methods of cryopreservation due to the fact that the cryopreserved cells need not be cultured or processed otherwise after they have been thawed.

Also provided herein are cellular compositions comprising cells that have been cryopreserved in accordance with the described methods.

### 6.1 Methods of Cryopreservation

In one aspect, provided herein are methods for preparing cryopreserved encapsulated cells suitable for administration to subjects immediately after the cryopreserved cells are thawed and encapsulated in alginate. In accordance with this aspect, the cryopreserved encapsulated cells are suspended in liquid alginate prior to their cryopreservation and encapsulated immediately after being thawed. Alternatively, the cryopreserved cells are suspended in liquid alginate immediately after being thawed and subsequently encapsulated. In each case, the encapsulated cells then can be administered to a subject without any additional culturing, e.g., the cells are used immediately after encapsulation. The cells used in such methods may comprise any cells described in Section 6.1.2, infra.

In a specific embodiment, provided herein is a method for preparing a population of encapsulated cells suitable for administration to a subject, said method comprising: (i) obtaining a population of cells; (ii) adding to said population of cells a cryopreservation solution comprising liquid alginate to produce a cell/liquid alginate composition; (iii) cryopreserving said cell/liquid alginate composition; (iv) thawing said cell/liquid alginate composition; and (v) encapsulating said cell/liquid alginate composition. In certain embodiments, the cells are used after thawing and subsequent encapsulation without any additional culturing, e.g., the cells are used immediately after encapsulation. In a specific embodiment, the cells used in the methods are stem cells. In another specific embodiment, the cells used in the method are stem cells isolated or derived from placental tissue (including the umbilical cord).

In another specific embodiment, provided herein is a method for preparing a population of encapsulated cells suitable for administration to a subject, said method comprising: (i) combining a population of cells and a cryopreservation solution comprising liquid alginate to produce a cell/liquid alginate composition; (ii) cryopreserving said cell/liquid alginate composition; (iii) thawing said cell/liquid alginate composition; and (iv) encapsulating said cell/liquid alginate composition. In certain embodiments, the cells are used after thawing and subsequent encapsulation without any additional culturing, e.g., the cells are used immediately after encapsulation. In a specific embodiment, the cells used in the methods are stem cells. In another specific embodiment, the cells used in the method are stem cells isolated or derived from placental tissue (including the umbilical cord).

In another specific embodiment, provided herein is a method for administering a population of encapsulated cells to a subject, said method comprising: (i) obtaining a population of cells; (ii) adding to said population of cells a cryopreservation solution comprising liquid alginate to produce a cell/liquid alginate composition; (iii) cryopreserving said cell/liquid alginate composition; (iv) thawing said cryopreserved cell/liquid alginate composition; (v) encapsulating said cell/liquid alginate composition; and (vi) administering the encapsulated cell/liquid alginate composition to a subject, wherein said administration is performed in the absence of culturing the cells in said cell/liquid alginate composition after the encapsulation of step (v). In a specific embodiment, the cells used in the methods are stem cells. In another specific embodiment, the cells used in the method are stem cells isolated or derived from placental tissue (including the umbilical cord).

In another specific embodiment, provided herein is a method for administering a population of encapsulated cells to a subject, said method comprising: (i) combining a population of cells and a cryopreservation solution comprising liquid alginate to produce a cell/liquid alginate composition; (ii) cryopreserving said cell/liquid alginate composition; (iii) thawing said cryopreserved cell/liquid alginate composition; (iv) encapsulating said cell/liquid alginate composition; and (v) administering the encapsulated cell/liquid alginate composition to a subject, wherein said administration is performed in the absence of culturing the cells in said cell/liquid alginate composition after the encapsulation of step (iv). In a specific embodiment, the cells used in the methods are stem cells. In another specific embodiment, the cells used in the method are stem cells isolated or derived from placental tissue (including the umbilical cord).

In another specific embodiment, provided herein is a method for preparing a population of encapsulated cells suitable for administration to a subject, said method comprising: (i) obtaining a population of cells; (ii) cryopreserving said cells in a cryopreservation solution; (iii) thawing the cryopreserved cells; (iv) adding to the thawed cells a solution comprising liquid alginate to produce a cell/liquid alginate composition; and (v) encapsulating said cell/liquid alginate composition. In certain embodiments, the cells are used after thawing and subsequent encapsulation without any additional culturing, e.g., the cells are used immediately after encapsulation. In a specific embodiment, the cells used in the methods are stem cells. In another specific embodiment, the cells used in the method are stem cells isolated or derived from placental tissue (including the umbilical cord).

In another specific embodiment, provided herein is a method for preparing a population of encapsulated cells suitable for administration to a subject, said method comprising: (i) cryopreserving a population of cells in a cryopreservation solution; (ii) thawing the cryopreserved cells; (iii) adding to the thawed cells a solution comprising liquid alginate to produce a cell/liquid alginate composition; and (iv) encapsulating said cell/liquid alginate composition. In certain embodiments, the cells are used after thawing and subsequent encapsulation without any additional culturing, e.g., the cells are used immediately after encapsulation. In a specific embodiment, the cells used in the methods are stem cells. In another specific embodiment, the cells used in the method are stem cells isolated or derived from placental tissue (including the umbilical cord).

In another specific embodiment, provided herein is a method for administering a population of encapsulated cells to a subject, said method comprising: (i) obtaining a population of cells; (ii) cryopreserving said cells in a cryopreservation solution; (iii) thawing the cryopreserved cells; (iv) adding to the thawed cells a solution comprising liquid alginate to produce a cell/liquid alginate composition; (v) encapsulating said cell/liquid alginate composition; and (vi) administering the encapsulated cell/liquid alginate composition to a subject, wherein said administration is performed in the absence of culturing the cells in said cell/liquid alginate composition after the encapsulation of step (v). In a specific embodiment, the cells used in the methods are stem cells. In another specific embodiment, the cells used in the method are stem cells isolated or derived from placental tissue (including the umbilical cord).

In another specific embodiment, provided herein is a method for administering a population of encapsulated cells to a subject, said method comprising: (i) cryopreserving a population of cells in a cryopreservation solution; (ii) thawing the cryopreserved cells; (iii) adding to the thawed cells a solution comprising liquid alginate to produce a cell/liquid alginate composition; (iv) encapsulating said cell/liquid alginate composition; and (v) administering the encapsulated cell/liquid alginate composition to a subject, wherein said administration is performed in the absence of culturing the cells in said cell/liquid alginate composition after the encapsulation of step (iv). In a specific embodiment, the cells used in the methods are stem cells. In another specific embodiment, the cells used in the method are stem cells isolated or derived from placental tissue (including the umbilical cord).

In another aspect, provided herein are methods for preparing cryopreserved cells suitable for administration to subjects immediately after the cryopreserved cells are thawed. In accordance with this aspect, the cryopreserved cells are suspended in liquid alginate prior to their cryopreservation and can be administered to a subject immediately after thawing, without the requirement that the encapsulated cells be cultured prior to the administration, e.g., the cells can be used immediately after being thawed. The cells used in such methods may comprise any cells described in Section 6.1.2, infra.

In a specific embodiment, provided herein is a method for preparing a population of cells suitable for administration to a subject, said method comprising: (i) obtaining a population of cells; (ii) adding to said population of cells a cryopreservation solution comprising liquid alginate to produce a cell/liquid alginate composition; (iii) cryopreserving said cell/liquid alginate composition, wherein the cells in the cryopreserved cell/liquid alginate composition can be used after thawing without any additional culturing, e.g., the cells can be used immediately after being thawed. In a specific embodiment, the cells used in the methods are stem cells. In another specific embodiment, the cells used in the method are stem cells isolated or derived from placental tissue (including the umbilical cord).

In another specific embodiment, provided herein is a method for preparing a population of cells suitable for administration to a subject, said method comprising: (i) combining a population of cells and a cryopreservation solution comprising liquid alginate to produce a cell/liquid alginate composition; and (ii) cryopreserving said cell/liquid alginate composition, wherein the cells in the cryopreserved cell/liquid alginate composition can be used after thawing without any additional culturing, e.g., the cells can be used immediately after being thawed. In a specific embodiment, the cells used in the methods are stem cells. In another specific embodiment, the cells used in the method are stem cells isolated or derived from placental tissue (including the umbilical cord).

In another specific embodiment, provided herein is a method for administering a population of cells to a subject, said method comprising: (i) combining a population of cells and a cryopreservation solution comprising liquid alginate to produce a cell/liquid alginate composition; (ii) cryopreserving the cell/liquid alginate composition; (iii) thawing the cell/liquid alginate composition; and (iv) administering the cell/liquid alginate composition to a subject, wherein said administration is performed in the absence of culturing the cells in said cell/liquid alginate composition after the thawing of step (iii). In a specific embodiment, the cells used in the methods are stem cells. In another specific embodiment, the cells used in the method are stem cells isolated or derived from placental tissue (including the umbilical cord).

In another specific embodiment, provided herein is a method for administering a population of encapsulated cells to a subject, said method comprising: (i) obtaining a population of cells; (ii) adding to said population of cells a cryopreservation solution comprising liquid alginate to produce a cell/liquid alginate composition; (iii) cryopreserving the cell/liquid alginate composition; (iv) thawing the cell/liquid alginate composition; and (v) administering the cell/liquid alginate composition to a subject, wherein said administration is performed in the absence of culturing the cells in said cell/liquid alginate composition after the thawing of step (iv). In a specific embodiment, the cells used in the methods are stem cells. In another specific embodiment, the cells used in the method are stem cells isolated or derived from placental tissue (including the umbilical cord).

6.1.1 Encapsulated Cell Populations

Further provided herein are populations of encapsulated cells generated in accordance with the methods described herein. Such encapsulated cells can be used for any desired purpose, e.g., for therapeutic purposes. In specific embodiments, the encapsulated cells are used as described in Section 6.3.

In a specific embodiment, provided herein is a population of encapsulated cells, wherein said cells are generated according to the following method: (i) combining cells and a cryopreservation solution comprising liquid alginate to produce a cell/liquid alginate composition; (ii) cryopreserving said cell/liquid alginate composition; (iii) thawing said cell/liquid alginate composition; and (iv) encapsulating said cell/liquid alginate composition. In a specific embodiment, the cells are stem cells. In another specific embodiment, the cells are stem cells isolated or derived from placental tissue (including the umbilical cord).

In another specific embodiment, provided herein is a population of encapsulated cells, wherein said cells are generated according to the following method: (i) cryopreserving cells in a cryopreservation solution; (ii) thawing the cryopreserved cells; (iii) adding to the thawed cells a solution comprising liquid alginate to produce a cell/liquid alginate composition; and (iv) encapsulating said cell/liquid alginate composition. In a specific embodiment, the cells are stem cells. In another specific embodiment, the cells are stem cells isolated or derived from placental tissue (including the umbilical cord).

In another specific embodiment, provided herein is a population of cells, wherein said cells are generated according to the following method: (i) combining a population of cells and a cryopreservation solution comprising liquid alginate to produce a cell/liquid alginate composition; and (ii) cryopreserving said cell/liquid alginate composition, and wherein the cells in the cryopreserved cell/liquid alginate composition can be used after thawing without any additional culturing, e.g., the cells can be used immediately after being thawed. In a specific embodiment, the cells are stem cells. In another specific embodiment, the cells are stem cells isolated or derived from placental tissue (including the umbilical cord).

6.1.2 Cells

Any cell type can be used in accordance with the methods described herein, including primary cells isolated directly from subjects and cell lines known to those of skill in the art. That is, the cells used in the described methods may be isolated or derived from any known tissue including connective tissue, epithelial tissue, muscle tissue, and/or nervous tissue. Sources of cells that can be used in accordance with the methods described herein include, but are not limited to, placenta (including the umbilical cord), bone marrow, stroma, mesenchyme, skin, bone, blood, lung, liver, brain, kidney, gall bladder, bladder, heart, spleen, stomach, pancreas, testicle, ovary, colon, small intestine, and large intestine.

In certain embodiments, the cells used in the methods described herein are stem cells, e.g., stem cells isolated or derived from placental tissue (including the umbilical cord), and mesenchymal stem cells (e.g., bone marrow-derived mesenchymal stem cells). Exemplary methods for obtaining stem cells isolated or derived from placental tissue (including the umbilical cord) are described in U.S. Pat. No. 7,468,276, U.S. Patent Application Publication No. 2007/0275362, and U.S. Patent Application Publication No. 2010/0124569, the disclosures of which are incorporated herein by reference in their entireties.

In a specific embodiment, the cells or cell populations used in the methods described herein are placental stem cells isolated from placenta. Placental stem cells and placenta stem cell populations are described in detail in, for example, U.S. Pat. No. 7,468,276, and in U.S. Patent Application Publication No. 2007/0275362, the disclosures of which are incorporated herein by reference in their entireties.

Placental stem cells are CD10⁺, CD34⁻, CD105⁺, CD200⁺ placental stem cells. In another specific embodiment, said placental stem cells express CD200 and do not express HLA-G; or express CD73, CD 105, and CD200; or express CD200 and OCT-4; or express CD73 and CD105 and do not express HLA-G; or express CD73 and CD105 and facilitate the formation of one or more embryoid-like bodies in a population of placental cells comprising said stem cell when said population is cultured under conditions that allow for the formation of an embryoid-like body; or express OCT-4 and facilitate the formation of one or more embryoid-like bodies in a population of placental cells comprising said stem cell when said population is cultured under conditions that allow for the formation of an embryoid-like body. In yet other embodiments, said placental stem cells express one or more of CD44, CD90, HLA-ABC, or HLA-P; and/or do not express one or more of CD45, CD119, CD133, KDR, CD80, CD86, HLA-DR, SSEA3, SSEA4, or CD38.

In another specific embodiment, the cells or cell populations used in the methods described herein are stem cells isolated from placenta referred to as “amnion-derived adherent cells,” or “AMDACs.” Such cells and cell populations are described in detail in, for example, U.S. Patent Application Publication No. 2010/0124569, the disclosure of which is incorporated herein by reference in its entirety.

AMDACs may be identified by different combinations of cellular and genetic markers. In a specific embodiment, for example, AMDACs are OCT-4⁻ as determinable by reverse-transcriptase-polymerase chain reaction (RT-PCR). In another embodiment, AMDACs are CD49f⁺, as determinable by flow cytometry. In yet another embodiment, AMDACs are OCT-4⁻ and CD49f⁺ as determinable by RT-PCR and flow cytometry, respectively. In still another embodiment, the AMDACs are CD49f⁺, CD105⁺, and CD200⁺ as determinable by immunolocalization, e.g., flow cytometry. In another embodiment, the AMDACs are OCT-4⁻ as determinable by RT-PCR and CD49f⁺, CD105⁺, and CD200⁺ as determinable by immunolocalization, e.g., flow cytometry. In another specific embodiment, AMDACs are positive for VEGFR1/Flt-1 (vascular endothelial growth factor receptor 1) and/or CD309 (also known as vascular endothelial growth factor receptor 2 (VEGFR2)/KDR), as determinable by immunolocalization, e.g., flow cytometry. In another specific embodiment, AMDACs are CD90⁺ and/or CD117⁻ as determinable by flow cytometry, and/or HLA-G⁻, as determinable by RT-PCR. In another specific embodiment, said AMDACs are OCT-4⁻ and HLA-G⁻, as determinable by RT-PCR, and CD49f⁺, CD90⁺, CD105⁺, and CD117⁻ as determinable by flow cytometry. In another specific embodiment, any of the above AMDACs are additionally one or more of CD9⁺, CD10⁺, CD44⁺, CD54⁺, CD98⁺, Tie-2⁺ (angiopoietin receptor), TEM-7⁺ (tumor endothelial marker 7), CD31⁻, CD34⁻, CD45⁻, CD133⁻, CD143⁻, CD146⁻, or CXCR4⁻ (chemokine (C—X—C motif) receptor 4) as determinable by immunolocalization, e.g., flow cytometry. In another specific embodiment, any of the above AMDACs are additionally CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, and CXCR4⁻ as determinable by immunolocalization, e.g., flow cytometry. In another specific embodiment, the AMDACs are GFAP⁺ as determinable by a short-term neural differentiation assay. In another specific embodiment, the AMDACs are beta-tubulin III (Tuj1)⁺ as determinable by a short-term neural differentiation assay.

In another specific embodiment, the cells used in the methods described herein are mesenchymal stem cells or “mesenchymal-like” stem cells. In a specific embodiment, the mesenchymal stem cells are bone marrow-derived mesenchymal stem cells.

6.1.3 Encapsulation

Any method known in the art for encapsulating cells suspended in liquid alginate, e.g., by exposing the cell/liquid alginate suspension to divalent cations, can be used in accordance with the methods described herein. Generally, methods for encapsulation of cells in alginate that are encompassed herein comprise suspending cells in liquid alginate to form a cell/liquid alginate suspension; dispersing the cell/liquid alginate suspension, e.g., as droplets in a drop-wise fashion (e.g., via a syringe); and exposing the dispersed suspension, e.g., droplets of the cell/liquid alginate suspension, to a solution comprising divalent cations (e.g., Calcium, Barium, Copper, Zinc or Strontium) which results in cross-linking of the alginate polymers in the cell/liquid alginate suspension and thus formation of encapsulated cells. The encapsulation methods encompassed herein do not include methods wherein cell/liquid alginate suspensions are merely frozen in alginate.

In certain embodiments, beads are formed when cells are encapsulated in alginate. In specific embodiments, the beads formed when cells are encapsulated in alginate form a hydrogel structure. Beads formed when cells are encapsulated in alginate can be formed so that their size meets a desired need. In certain embodiments, the beads formed when cells are encapsulated in alginate are about 100 μm, about 200 μm, about 300 μm, about 400 μm, about 500 μm, about 600 μM, or about 700 μm in size. In other embodiments, the beads formed when cells are encapsulated in alginate are about 100-200 μm, about 100-300 μm, about 200-400 μm, about 200-500 μM, about 300-500 μm, about 300-600 μm, about 400-600 μm, or about 500-600 μm, about 500-700 μm size. In a specific embodiment, the beads formed when cells are encapsulated in alginate are less than 500 μm in size.

The amount of alginate in the cell/liquid alginate solutions can be determined based on the desired result, e.g., the desired viscosity of alginate solution. In certain embodiments, the amount of alginate in the cell/liquid alginate solution is less than 0.5%. In other embodiments, the amount of alginate in the cell/liquid alginate solution is about 0.5%, about 0.6%, about 0.7%, about 0.75%, about 0.8%, about 0.9%, about 1.0%, about 1.1%, about 1.2%, about 1.3%, about 1.4%, about 1.5%. In other embodiments, the amount of alginate in the cell/liquid alginate solution is greater than 1.5%. In other embodiments, the amount of alginate in the cell/liquid alginate solution is from about 0.5% to about 1.0%, from about 0.75% to about 1.5%, or from about 1.0% to about 1.5%. In a specific embodiment, the amount of alginate in the cell/liquid alginate solution is 0.75%. In specific embodiments, the amount of alginate in the cell/liquid alginate solution is sufficient to yield a viscosity of ≧0.009 Pa·s.

The dispersement of the cell/liquid alginate solution can be accomplished by any means known to those skilled in the art, including, without limitation, immersion, submersion, spraying, dispersing as droplets, e.g., via a syringe, electrostatic generation, or atomization.

The divalent cations used for cross-linking the alginate and thus encapsulating the cells can be any divalent cation known in the art to accomplish the technique. In certain embodiments, the divalent cation used to cross-link the alginate in the cell/liquid alginate solution is calcium chloride (CaCl₂), barium chloride (BaCl₂), stromium chloride (SrCl₂), copper chloride (CuCl₂), or zinc chloride (ZnCl₂). In a specific embodiment, the divalent cation used to cross-link the alginate in the cell/liquid alginate solution is calcium chloride (CaCl₂). In certain embodiments, the solution of divalent cation comprises about 0.5%, about 0.75%, about 1.0%, about 1.25%, about 1.5%, about 1.75%, or about 2.0% divalent cation. In a specific embodiment, the solution of divalent cation comprises 1.5% divalent cation, e.g., CaCl₂.

6.1.4 Cryopreservation

Any method known in the art for cryopreserving cells can be used in accordance with the methods described herein. Cells can be cryopreserved in a cryopreservation solution described herein in small containers (e.g., ampoules); in bags suitable for cryopreservation; or in any other suitable container for cryopreservation. In some embodiments, cells are cryopreserved in commercially available cryopreservation medium, for example commercially available cell freezing medium, e.g., cell freezing medium identified by Sigma Aldrich catalog numbers C2695, C2639 (Cell Freezing Medium-Serum-free 1×, not containing DMSO) or C6039 (Cell Freezing Medium-Glycerol 1× containing Minimum Essential Medium, glycerol, calf serum and bovine serum), Lonza PROFREEZE™ 2× Medium, or Plasmalyte.

In some embodiments, the cells processed in accordance with the methods described herein, whether in encapsulated or unencapsulated form, may be cryopreserved in a cryopreservation solution comprising one or more components, such as human serum albumin (HSA). In certain embodiments, the solution comprises about 5%, about 10%, about 15%, about 20%, about 25%, about 30%, about 35%, about 40%, about 45%, about 50%, about 55%, or about 60% HSA. In other embodiments, the solution comprises about 5% to about 25%, about 10% to about 30%, about 20% to about 40%, about 30% to about 50%, about 40% to about 60%, or about 50% to about 60% HSA. In a specific embodiment, the solution comprises 40% HSA.

In other embodiments, the cells processed in accordance with the methods described herein, whether in encapsulated or unencapsulated form, may be cryopreserved in a cryopreservation solution comprising one or more cryoprotectants, e.g., DMSO. In certain embodiments, the solution comprises about 1%, about 1.5%, about 2%, about 2.5%, about 3%, about 4%, about 5%, about 6%, about 7%, about 8%, about 9%, or about 10% DMSO. In other embodiments, the solution comprises about 1% to about 3%, about 2% to about 4%, about 3% to about 5%, about 4% to about 6%, about 5% to about 7%, about 6% to about 8%, about 7% to about 9%, or about 8% to about 10% DMSO. In a specific embodiment, the solution comprises 2.5% DMSO. In another specific embodiment, the solution comprises 5% DMSO.

In other embodiments, the cells processed in accordance with the methods described herein, whether in encapsulated or unencapsulated form, may be cryopreserved in a cryopreservation solution comprising one or more solutions for use in storing cells, such as HypoThermosol® (BioLife Solutions, Bothell, Wash.)). In certain embodiments, the solution comprises about 25%, about 30%, about 35%, about 40%, about 45%, about 50%, about 55%, about 60%, about 65%, or about 70% HypoThermosol®. In other embodiments, the solution comprises about 25% to about 50%, about 40% to about 60%, about 50% to about 60%, about 50% to about 70%, or about 60% to about 70% HypoThermosol®. In a specific embodiment, the solution comprises 55% HypoThermosol®. In another specific embodiment, the solution comprises57.5% HypoThermosol®.

In other embodiments, the cells processed in accordance with the methods described herein, whether in encapsulated or unencapsulated form, may be cryopreserved in a cryopreservation solution comprising one or more excipients, such as dextran, starch, glucose, lactose, sucrose, gelatin, silica gel, glycerol monostearate, sodium chloride, glycerol, propylene, and/or glycol. In addition, the cells processed in accordance with the methods described herein, whether in encapsulated or unencapsulated form, may be cryopreserved in a cryopreservation solution comprising certain media, e.g., PBS or DMEM.

In a specific embodiment, the cells processed in accordance with the methods described herein are cryopreserved in a cryopreservation solution comprising 40% HSA, 5% DMSO, and 55% HypoThermosol®. In a specific embodiment, the cells in the solution are encapsulated and cryopreserved in the solution. In another specific embodiment, the cells in the solution are not encapsulated and cryopreserved in the solution.

Cells may be cooled, for example, at about 1° C./min during cryopreservation. In some embodiments, the cryopreservation temperature is about −80° C. to about −180° C., or about −125° C. to about −140° C. Cryopreserved cells can be transferred to vapor phase of liquid nitrogen prior to thawing for use. In some embodiments, for example, once the cells have reached about −80° C., they are transferred to a liquid nitrogen storage area. Cryopreservation can also be done using a controlled-rate freezer. Cryopreserved cells may be thawed, e.g., at a temperature of about 25° C. to about 40° C., and typically at a temperature of about 37° C.

### 6.2 Pharmaceutical Compositions

Provided herein are pharmaceutical compositions comprising cells that have been processed in accordance with the methods provided herein, including the cells in the cellular compositions described in Section 6.1.1, supra. In specific embodiments, the cells in the pharmaceutical compositions provided herein are a cell type described in Section 6.1.2, supra.

The pharmaceutical compositions provided herein may comprise a population of encapsulated cells or a population of unencapsulated cells in a cell/liquid alginate solution formulated for in vivo administration. In some embodiments, the cells are cryopreserved in a cryopreservation solution that represents an acceptable pharmaceutical composition, thus allowing the cells to be directly administered to a subject after thawing, for example, the cells are cryopreserved in a cryopreservation solution comprising one or more of the components described in Section 6.1.4. In other embodiments, the cells are cryopreserved in a cryopreservation solution that represents an acceptable pharmaceutical composition, wherein the solution comprises alginate, and wherein the cells are encapsulated immediately after being thawed, followed by direct administration of the encapsulated cells to a subject.

In one embodiment, the cells in the compositions provided herein are administered to a subject in the form of a composition comprising cells in a container. In a specific embodiment, the container is a bag, flask, vial, or jar. The cells can be removed from the container and administered to a subject using any appropriate means known in the art or described in Section 6.3.1, infra, e.g., injection. In certain embodiments, the container comprises about, at least, or at most 1×10² cells, 1×10³ cells, 1×10⁴ cells, 1×10⁵ cells, 1×10⁶ cells, 5×10⁶ cells, 1×10⁷ cells, 5×10⁷ cells, 1×10⁸ cells, 5×10⁸ cells, 1×10⁹ cells, 5×10⁹ cells, 1×10¹⁰ cells, or 1×10¹¹ cells. In a specific embodiment, the cells in the pharmaceutical composition are stem cells. In another specific embodiment, the cells in the pharmaceutical composition are placental stem cells. In another specific embodiment, the cells in the pharmaceutical composition are AMDACs.

In certain embodiments, the pharmaceutical compositions provided herein comprise populations of cells that comprise at least 25%, 30%, 35%, 40%, 45%, 50%, 55%, 60%, 65%, 70%, 75%, 80%, 85%, 90%, 95%, or 99% viable cells. In a specific embodiment, the pharmaceutical compositions provided herein comprise populations of cells that comprise at least 95% viable cells. In another specific embodiment, the pharmaceutical compositions provided herein comprise populations of cells that comprise at least 90% viable cells. In a specific embodiment, the pharmaceutical compositions provided herein comprise populations of cells that comprise at least 80% viable cells. In a specific embodiment, the pharmaceutical compositions provided herein comprise populations of cells that comprise at least 75% viable cells. In a specific embodiment, the pharmaceutical compositions provided herein comprise populations of cells that comprise at least 50% viable cells. In a specific embodiment, the pharmaceutical compositions provided herein comprise populations of cells that comprise at least 40% viable cells.

### 6.3 Uses

The cells processed in accordance with the methods described herein and pharmaceutical compositions thereof are useful for many purposes including, but not limited to, therapeutic uses. The cells and pharmaceutical compositions thereof are particularly useful due to the fact that they can be administered to a subject (i) after they have been thawed and subsequently encapsulated or (ii) after being thawed and without a subsequent encapsulation step. In each case, the cells and pharmaceutical compositions thereof are useful in that they can be administered to a subject without the need for any intervening culture steps after thawing and before administration, e.g., the cells can be administered immediately. In addition, the cells processed in accordance with the methods described herein and pharmaceutical compositions thereof are useful in that they can be locally administered to a subject. As such, provided herein are methods of administering (e.g., locally administering) cells processed in accordance with the methods described herein or pharmaceutical compositions thereof to subjects.

6.3.1 Dosages and Routes of Administration

Administration of cells (e.g., AMDACs or placental stem cells) processed in accordance with the methods described herein or a pharmaceutical composition thereof to a subject can be by any medically-acceptable route that is suitable for administration of the cells. In a specific embodiment, the cells are administered locally, e.g., at a particular site in the body of the subject that is relevant to the purpose of administration. In another specific embodiment the cells are administered by bolus injection. In another specific embodiment, the cells are administered intracranially. In another specific embodiment, said the cells are administered intramuscularly. In another specific embodiment, the cells are administered intraperitoneally. In another specific embodiment, the cells are administered intradermally, or subcutaneously. In another specific embodiment, the cells are administered subcutaneously. In another specific embodiment, the cells are administered intrasternally. In another specific embodiment, the cells are administered intrasynovially. In another specific embodiment, the cells are administered intraocularly. In another specific embodiment, the cells are administered intravitreally. In another specific embodiment, the cells are administered intracerebrally. In another specific embodiment, the cells are administered intracerebroventricularly. In another specific embodiment, the cells are administered intrathecally. In another specific embodiment, the cells are administered by intraosseous infusion. In another specific embodiment, the cells are administered intravesically. In another specific embodiment, the cells are administered transdermally. In another specific embodiment, the cells are administered intracisternally. In another specific embodiment, the cells are administered epidurally.

In another specific embodiment, the cells are administered once to a subject. In another specific embodiment, the cells are administered to a subject in two or more separate administrations. In a specific embodiment, the administration comprises administering between about 1×10² and 1×10³ cells per kilogram of a subject. In another specific embodiment, the administration comprises administering between about 1×10³ and 1×10⁴ cells per kilogram of a subject. In another specific embodiment, the administration comprises administering between about 1×10⁴ and 1×10⁵ cells per kilogram of a subject. In another specific embodiment, the administration comprises administering between about 1×10⁵ and 1×10⁶ cells per kilogram of a subject. In another specific embodiment, the administration comprises administering between about 1×10⁶ and 1×10⁷ cells per kilogram of a subject. In another specific embodiment, the administration comprises administering between about 1×10⁷ and 1×10⁸ cells per kilogram of a subject. In another specific embodiment, the administration comprises administering between about 1×10⁸ and 1×10⁹ cells per kilogram of a subject. In another specific embodiment, the administration comprises administering between about 1×10⁹ and 1×10¹⁰ cells per kilogram of a subject. In another specific embodiment, the administration comprises administering between about 1×10¹⁰ and 1×10¹¹ cells per kilogram of a subject. In other specific embodiments, the administration comprises administering between about 1×10⁴ and about 2×10⁴ cells per kilogram of a subject; between about 2×10⁴ and about 3×10⁴ cells per kilogram of a subject; between about 3×10⁴ and about 4×10⁴ cells per kilogram of a subject; between about 4×10⁴ and about 5×10⁴ cells per kilogram of a subject; between about 5×10⁴ and about 6×10⁴ cells per kilogram of a subject; between about 6×10⁴ and about 7×10⁴ cells per kilogram of a subject; between about 7×10⁴ and about 8×10⁴ cells per kilogram of a subject; between about 8×10⁴ and about 9×10⁴ cells per kilogram of a subject; or between about 9×10⁴ and about 1×10⁵ cells per kilogram of a subject. In other specific embodiments, the administration comprises administering between about 1×10⁵ and about 2×10⁵ cells per kilogram of a subject; between about 2×10⁵ and about 3×10⁵ cells per kilogram of a subject; between about 3×10⁵ and about 4×10⁵ cells per kilogram of a subject; between about 4×10⁵ and about 5×10⁵ cells per kilogram of a subject; between about 5×10⁵ and about 6×10⁵ cells per kilogram of a subject; between about 6×10⁵ and about 7×10⁵ cells per kilogram of a subject; between about 7×10⁵ and about 8×10⁵ cells per kilogram of a subject; between about 8×10⁵ and about 9×10⁵ cells per kilogram of a subject; or between about 9×10⁵ and about 1×10⁶ cells per kilogram of a subject. In other specific embodiments, the administration comprises administering between about 1×10⁶ and about 2×10⁶ cells per kilogram of a subject; between about 2×10⁶ and about 3×10⁶ cells per kilogram of a subject; between about 3×10⁶ and about 4×10⁶ cells per kilogram of a subject; between about 4×10⁶ and about 5×10⁶ cells per kilogram of a subject; between about 5×10⁶ and about 6×10⁶ cells per kilogram of a subject; between about 6×10⁶ and about 7×10⁶ cells per kilogram of a subject; between about 7×10⁶ and about 8×10⁶ cells per kilogram of a subject; between about 8×10⁶ and about 9×10⁶ cells per kilogram of a subject; or between about 9×10⁶ and about 1×10⁷ cells per kilogram of a subject. In a specific embodiment, the cells administered are stem cells. In another specific embodiment, the cells administered are placental stem cells. In another specific embodiment, the cells administered are AMDACs.

In another specific embodiment cells are administered to a subject as a single unit dose. In specific embodiments, a single unit dose of cells can comprise, in various embodiments, about, at least, or no more than 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more cells. In a specific embodiment, the cells administered as a single unit dose are stem cells. In another specific embodiment, the cells administered as a single unit dose are placental stem cells. In another specific embodiment, the cells administered as a single unit dose are AMDACs.

### 7. EXAMPLES

### 7.1 Example 1

### Viability of Encapsulated Placental Stem Cells

This Example demonstrates that placental stem cells are viable when encapsulated in alginate.

7.1.1 Storage at 25° C. and 37° C.

Placental stem cells (5×10⁵ cells/ml) were suspended in a 0.75% alginate solution and encapsulated by dripping the cell/liquid alginate solution into a solution of CaCl₂ (1.5% w/v). The encapsulated cells were stored at either 25° C. or 37° C. for seven days and cell viability was measured at various time points by labeling dead cells with propidium iodide and by labeling live cells with calcein acetomethoxy (AM).

The encapsulated placental stem cells remained viable for 7 days at 37° C. and for 2 days at 25° C.

7.1.2 Cold Storage

placental stem cells (5×10⁵ cells/ml and 2×10⁶ cells/ml) were suspended in a 0.75% alginate solution and encapsulated by dripping the cell/liquid alginate solution into a solution of CaCl₂ (1.5% w/v). HypoThermosol® (BioLife Solutions, Bothell, Wash.) storage solution was added to the encapsulated placental stem cells, and the cells were stored at either 4° C. or 37° C. for seven days (i.e., the cells were stored in 100% HypoThermosol®). Cell viability was measured at various time points by labeling dead cells with propidium iodide and by labeling live cells with calcein AM or by measuring the levels of ATP using the CellTiter-Glo® Luminescent Cell Viability Assay (Promega, Madison, Wis.), which allows for determination of the number of viable cells in culture based on quantitation of the ATP present, which signals the presence of metabolically active cells.

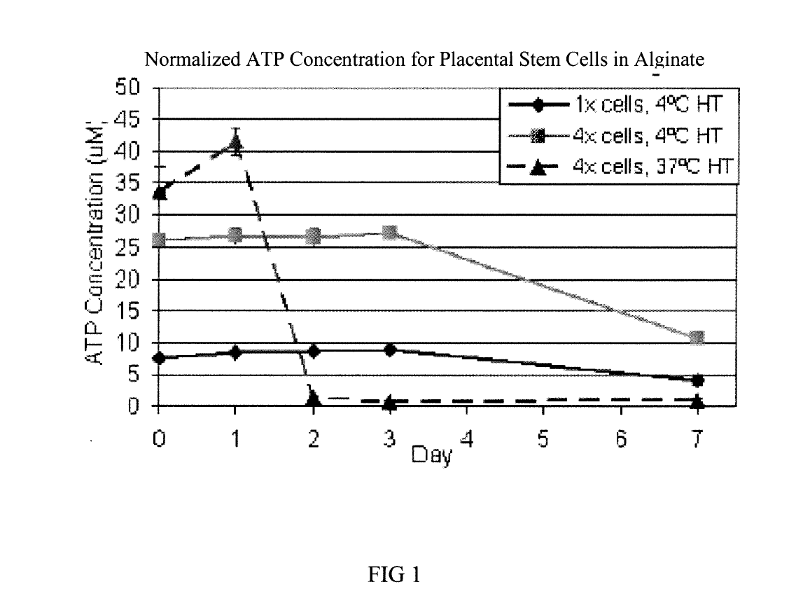

The encapsulated placental stem cells in HypoThermosol® cultured at 37° C. died after three days time, as expected. However, the encapsulated placental stem cells in HypoThermosol® cultured at 4° C. survived for up to 7 days. See FIG. 1.

### 7.2 Example 2

### Encapsulation of Cryopreserved Placental Stem Cells

This Example demonstrates that cryopreserved placental stem cells can be encapsulated after they have been thawed and remain viable without the need to culture the cells post-thaw.

7.2.1 Addition of Alginate Post-Thaw and Encapsulation Post-Thaw

Placental stem cells (7.5×10⁶ cells/ml) that had been cryopreserved in a cryopreservation solution comprising 40% HSA, 55% Dextran 40, and 5% DMSO were thawed and, without any intervening culture steps, were mixed with 1.5% alginate (in PBS) at a 1:1 ratio. The cells then were encapsulated as described in Example 1. Following encapsulation, and without any intervening culture steps, HypoThermosol® (BioLife Solutions, Bothell, Wash.) storage solution was added to the encapsulated placental stem cells, and the cells were stored at 4° C. in a syringe for seven days (i.e., the cells were stored in 100% HypoThermosol®). Viability of the encapsulated placental stem cells was analyzed using a Vi-CELL® Cell Viability Analyzer (Beckman Coulter, Brea, Calif.) in accordance with manufacturer's instructions. Immediately after encapsulation, the placental stem cells were approximately 80% viable. After 7 days in cold storage, approximately 45% of the placental stem cells were viable.

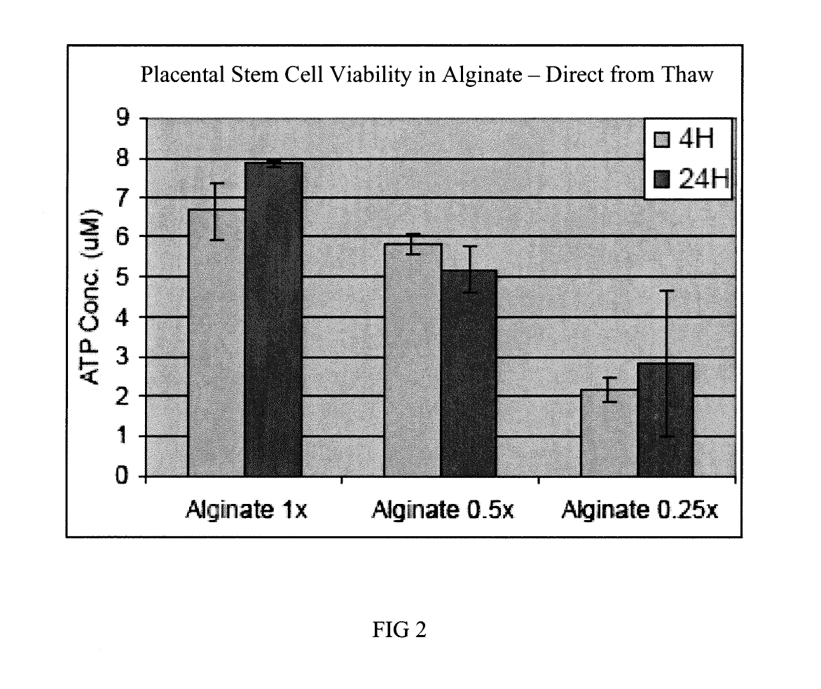

In a separate experiment, placental stem cells (7.5×10⁶ cells/ml) that had been cryopreserved in a cryopreservation solution comprising 40% HSA, 55% Dextran 40, and 5% DMSO were thawed and, without any intervening culture steps, were mixed with 1.5% alginate (in PBS) at a 1:1 ratio. The cells then were encapsulated as described in Example 1. Following encapsulation, and without any intervening culture steps, the encapsulated placental stem cells were diluted in growth medium at 3 separate dilutions (1×, 0.5×, and 0.25×) and viability was assessed at four hours and 24 hours post-encapsulation using the CellTiter-Glo® Luminescent Cell Viability Assay (Promega, Madison, Wis.). The encapsulated placental stem cells were viable at both time points, with viability remaining relatively constant between 4 and 24 hours post-encapsulation. See FIG. 2.

These experiments together demonstrate that cryopreserved placental stem cells can be suspended in alginate and subsequently encapsulated after they have been thawed, while retaining their viability post-thaw without the need to be cultured. The first experiment additionally demonstrates that placental stem cells that have been suspended in alginate and subsequently encapsulated immediately after thawing can be cold stored for short periods of time while retaining viability.

7.2.2 Addition of Alginate Pre-Cryopreservation and Encapsulation Post-Thaw

Placental stem cells (7.5×10⁶ cells/ml) were cryopreserved in a cryopreservation solution comprising 5% DMSO, 40% human serum albumin (HSA), and 55% alginate (1.5% concentration). The cryopreserved placental stem cells were subsequently thawed followed by encapsulation of the placental stem cells as described in Example 1. Viability of the encapsulated placental stem cells was assessed by ViCell as described above. The encapsulated placental stem cells retained greater than 95% viability for up to 7 hours post-encapsulation and approximately 90% viability at 24 hours post-encapsulation at 25° C.

This experiment demonstrates that placental stem cells cryopreserved in liquid alginate can be encapsulated after they have been thawed, and that the thawed, encapsulated cells retain their viability without the need to be cultured.

### Equivalents:

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

1. A method for preparing a population of encapsulated cells suitable for administration to a subject comprising:
(i) combining a population of cells and a cryopreservation solution comprising liquid alginate to produce a cell/liquid alginate composition;
(ii) cryopreserving said cell/liquid alginate composition;
(iii) thawing said cell/liquid alginate composition; and
(iv) encapsulating the cells in said cell/liquid alginate composition.

2. A method for preparing a population of cells suitable for administration to a subject comprising:
(i) combining a population of cells and a cryopreservation solution comprising liquid alginate to produce a cell/liquid alginate composition;
(ii) cryopreserving said cell/liquid alginate composition; and
(iii) thawing said cell/liquid alginate composition,
wherein the cells in the cryopreserved cell/liquid alginate composition can be immediately administered to the subject after thawing.

3. A method for administering the encapsulated cells of claim 1 to a subject comprising:
wherein said administration is performed in the absence of culturing the cells in said cell/liquid alginate composition after the encapsulation of step (iv).

4. A method for administering the cells of claim 2 to a subject comprising:
wherein said administration is performed in the absence of culturing the cells in said cell/liquid alginate composition after the thawing of step (iii).

5. The method of claim 1, wherein the cells are stem cells.

6. The method of claim 5, wherein the stem cells are isolated or derived from placental tissue.

7. The method of claim 6, wherein the stem cells are placental stem cells or amnion derived adherent cells (AMDACs).

8. The method of claim 1, wherein the cryopreservation solution further comprises one or more of DMSO, human serum albumin, dextran, or HypoThermosol®.

9. A cellular composition comprising a population of cells, wherein said cells are generated according to one of the following methods:
(a)(i) combining cells and a cryopreservation solution comprising liquid alginate to produce a cell/liquid alginate composition; (ii) cryopreserving said cell/liquid alginate composition; (iii) thawing said cell/liquid alginate composition; and (iv) encapsulating said cell/liquid alginate composition;
(b) (i) cryopreserving cells in a cryopreservation solution; (ii) thawing the cryopreserved cells; (iii) adding to the thawed cells a solution comprising liquid alginate to produce a cell/liquid alginate composition; and (iv) encapsulating said cell/liquid alginate composition; or
(c) (i) combining a population of cells and a cryopreservation solution comprising liquid alginate to produce a cell/liquid alginate composition; and (ii) cryopreserving said cell/liquid alginate composition, and wherein the cells in the cryopreserved cell/liquid alginate composition can be used after thawing without any additional culturing.

10. (canceled)

11. (canceled)

12. The method of claim 9, wherein the cells are stem cells.

13. The method of claim 12, wherein the stem cells are isolated or derived from placental tissue.

14. The method of claim 13, wherein the stem cells are placental stem cells or AMDACs.

15. The cellular composition of claim 9, wherein the cells are suspended in HypoThermosol®.

16. The method of claim 2, wherein the cells are stem cells.

17. The method of claim 2, wherein the cryopreservation solution further comprises one or more of DMSO, human serum albumin, dextran, or HypoThermosol®.
